(19) United States
(12) Patent Application Publication
(10) Pub. No.: US 2025/0241571 A1
(43) Pub. Date: Jul. 31, 2025

(54) WEARABLE DEPRESSIVE DISORDER RECOGNITION AND SEIZURE DETECTION DEVICE AND METHOD

(71) Applicant: Shenzhen University, Shenzhen (CN)

(72) Inventors: Yongpan ZOU, Shenzhen (CN); Kaishun WU, Shenzhen (CN); Jie ZHENG, Shenzhen (CN)

(73) Assignee: Shenzhen University, Shenzhen (CN)

(21) Appl. No.: 19/020,958

(22) Filed: Jan. 14, 2025

(30) Foreign Application Priority Data
Jan. 26, 2024 (CN) 202410109248.5

(51) Int. Cl. A61B 5/16; A61B 5/00

(52) U.S. Cl. CPC A61B 5/165; A61B 5/4094; A61B 5/681; A61B 5/7267

(57) ABSTRACT

Disclosed are a wearable depressive disorder recognition and seizure detection method and device. The method includes the following steps of: acquiring multi-modal data of a target user by using a wearable device; inputting the multi-modal data into a trained depressive disorder recognition model to determine whether the target user is a depression disorder person or a normal person; and inputting the corresponding multi-modal data to a trained depressive disorder seizure detection model to determine whether the target user is in a seizure state in the case of a determined depressive disorder person.

### CROSS-REFERENCE TO RELATED APPLICATIONS

The application claims priority to Chinese Patent Application No. 202410109248.5, filed on Jan. 26, 2024, the entire contents of which are incorporated herein by reference.

### TECHNICAL FIELD

The application relates to the technical field of electronic information, in particular to a wearable depressive disorder recognition and seizure detection device and method.

### BACKGROUND

Researchers have used various sensors to recognize depressive disorders, such as capturing facial information using a camera, extracting features from audio signals, analyzing text semantics, and using physiological signals such as electroencephalograms, electrocardiograms, and the like. However, such methods have the problems of privacy disclosure, environmental dependence and high cost. In recent years, wearable devices such as smart watches have emerged, with built-in rich sensors, providing more convenient and ubiquitous data acquisition methods, which can reflect the user's physiological state and emotional fluctuations. Despite the popularity and portability of wearable devices, there is still room for improvement in their accuracy and application scenarios.

In the prior art, schemes for recognizing depressive disorders are mainly divided into two categories. One is to use the user's image, audio, text or multi-modal combination of these modalities to recognize depressive disorders, but the data of these modalities are facing privacy leaks, and the impact of the environment leads to the reduction of system performance. The other is to use ubiquitous wearable devices or medical equipment to collect physiological data of users for assessment and recognition of depressive disorders. This approach typically collects only user physiological data for depressive disorder recognition. For example, the ubiquitous wearable device is used to obtain user physical information, such as user activity, sleep and heart rate related data. When physiological data such as electroencephalogram (EEG) and electrocardiogram (ECG) are collected by medical equipment to recognize depressive disorders, single-modality data are generally used, and the cost of medical equipment is high, which limits their application in large-scale popularization, and lacks universal use and convenience.

According to analysis, there are few studies on depressive disorder recognition based on multi-modal feature fusion of physiological data collected by wearable devices at home and abroad, and user personalization is generally not supported. In addition, there is no protocol for detecting the seizure status of patients with depressive disorders. For example, in 2020, Pedrelli Paola et al. used Empatica E4's wristband device and smartphone sensors to monitor the behavior and physiology of patients with major depressive disorder for 17 weeks. Clinical interviews and the Hamilton Depression Rating Scale (HDRS) were then used during this period to assess the feasibility of the severity of the patient's depressive disorder. The scheme designs three models to combine the patient's behavior and physiological characteristics to obtain the estimated value of HDRS score, and carries out correlation analysis with the HDRS assessed by doctors, and the results show that there is a high correlation. For another example, in 2019, Xu Xuhai et al. used multiple sensors of smartphones to provide user context features at the same time, used Fitbit wristband devices to collect user sleep data, and then mined these time series mobile data through association rules to extract deep context features. These features are then used to detect depressive disorder through traditional machine learning methods.

Through analysis, the existing scheme mainly has the following defects.

Firstly, the cross-user situation is not considered, that is, the situation of new users is not considered for the model, and only a corresponding depressive disorder recognition model is established for each modal data.

Secondly, when using image, audio, and text data, there will be privacy leakage issues, and recognition performance is also affected by the environment.

Thirdly, using the data collected by medical professional equipment to diagnose users, this system costs more, and only uses single mode physiological data, so the reliability of the system is low.

Fourthly, the existing wearable device-based depressive disorder recognition scheme does not support personalized users and cannot well adapt to new users.

Lastly, the existing schemes are all used for researching the single problem of depressive disorder recognition, but do not detect the seizure state of a depressive disorder patient, and do not combine the depressive disorder recognition with the depressive disorder seizure state, so that the patient diagnosed with the depressive disorder cannot further know the state of the patient.

### SUMMARY

The application aims to overcome the defects of the prior art and provide a wearable depressive disorder recognition and seizure detection method and device.

According to a first aspect of the present application, a wearable depressive disorder recognition and seizure detection method is provided. The method comprises the following steps of:
acquiring multi-modal data of a target user by using a wearable device;
inputting the multi-modal data into a trained depressive disorder recognition model to determine whether the target user is a depression disorder person or a normal person; and

inputting the corresponding multi-modal data to a trained depressive disorder seizure detection model to determine whether the target user is in a seizure state in the case of a determined depressive disorder person.

According to a second aspect of the present application, a wearable depressive disorder recognition and seizure detection device is provided. The device comprises:
a wearable device: for acquiring multi-modal data of a target user;
a recognition module for inputting the multi-modal data into a trained depressive disorder recognition model to determine whether the target user is a depression disorder person or a normal person; and
a detection module: inputting the corresponding multi-modal data to a trained depressive disorder seizure detection model to determine whether the target user is in a seizure state in the case of a determined depressive disorder person.

Compared with the prior art, the application has the advantages that the provided depressive disorder recognition and seizure detection scheme can be realized on the wearable device, wherein the wearable device is utilized to automatically collect physiological data in different modes for fusion feature, and the fused feature is transmitted to the depression disorder recognition model to obtain a recognition result, the physiological data of the recognized physiological data depressive disorder patient is transmitted to the depressive disorder seizure detection model to determine whether the patient is in a seizure state, so that the current state of the user can be known in real time, and extreme harmful behaviors are effectively avoided.

Other features and advantages of the present application will become apparent from the following detailed description of exemplary embodiments of the application with reference to the figures.

### BRIEF DESCRIPTION OF THE DRAWINGS

The figures, which are incorporated in and constitute a part of the specification, illustrate embodiments of the application and, together with the description, serve to explain the principles of the application. In the drawings:

FIG. 1 illustrates a flowchart of a wearable depressive disorder recognition and seizure detection method according to an embodiment of the present application;

FIG. 2 illustrates an overall process diagram of a wearable depressive disorder recognition and seizure detection method according to an embodiment of the present application;

FIG. 3 illustrates a schematic diagram of a framework of a wearable device according to an embodiment of the present application;

FIG. 4 illustrates an appearance diagram of a framework of a wearable device according to an embodiment of the present application;

FIG. 5 illustrates a schematic diagram of an internal structure of a wearable device according to an embodiment of the present application;

FIG. 6 illustrates a schematic diagram of a backbone network model structure according to an embodiment of the present application;

FIG. 7 illustrates a schematic diagram of a process of training a depressive disorder recognition model according to an embodiment of the present application;

FIG. 8 illustrates a schematic diagram of a process of training a depressive disorder seizure detection model according to an embodiment of the present application;

FIG. 9 illustrates a schematic diagram of a process of depressive disorder recognition and seizure detection using a wearable device according to an embodiment of the present application;

FIG. 10 illustrates a schematic diagram of the accuracy rate of the depressive disorder recognition test for different target users according to an embodiment of the present application; and

FIG. 11 illustrates a schematic diagram of the accuracy rate of the depressive disorder seizure detection for different target users according to an embodiment of the present application.

### DETAILED DESCRIPTION OF THE EMBODIMENTS

Various exemplary embodiments of the present application will now be described in detail with reference to the figures. It should be noted that the relative arrangement of parts and steps, the numerical expressions, and the numerical values set forth in these embodiments do not limit the scope of the application unless specifically stated otherwise.

The following description of at least one exemplary embodiment is merely illustrative in nature and is in no way intended to limit the application, its application, or uses.

Techniques, methods, and devices known to those of ordinary skill in the pertinent art may not be discussed in detail, but should be considered part of the specification, where appropriate.

In all instances shown and discussed herein, any particular value is to be construed as merely illustrative and not restrictive. Thus, other examples of example embodiments may have different values.

It should be noted that like reference numbers and letters refer to like items in the following figures, and therefore, once an item is defined in one figure, it needs not be further discussed in subsequent figures.

In general, according to the present application, a ubiquitous and portable wearable device is used to collect a plurality of physiological signals and other data of a user for multi-modal feature fusion, so as to realize depressive disorder recognition and detection of the seizure state of a patient with depressive disorder. According to the application, the cost of data acquisition can be reduced, and the depressive disorder patient can be better monitored in real time, so that the extreme harmful behaviors of the patient can be effectively avoided. In addition, the present application uses unsupervised transfer learning and few-shot learning methods to solve the differences in physiological data between individual users, thereby achieving depressive disorder recognition and seizure detection that supports user personalization.

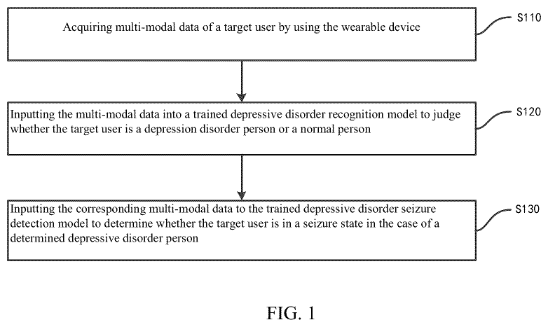

As shown in FIG. 1, the provided wearable depressive disorder recognition and seizure detection method comprises steps of: S110, acquiring multi-modal data of a target user by using a wearable device; S120, inputting the multi-modal data into a trained depressive disorder recognition model to determine whether the target user is a depression disorder person or a normal person; S130, inputting the corresponding multi-modal data to a trained depressive disorder seizure detection model to determine whether the target user is in a seizure state in the case of a determined depressive disorder person.

Correspondingly, the application also provides a wearable depressive disorder recognition and seizure detection device. The device comprises a wearable device, for acquiring multi-modal data of a target user; a recognition module, for inputting the multi-modal data into a trained depressive disorder recognition model to determine whether the target user is a depression disorder person or a normal person; and a detection module, for inputting the corresponding multi-modal data to a trained depressive disorder seizure detection model to determine whether the target user is in a seizure state in the case of a determined depressive disorder person.

It should be understood that the recognition module and the detection module may be embedded in the wearable device or independent of the wearable device, for example, the functions of the recognition module and the detection module are integrated into the wearable device, or integrated into the mobile terminal, the server, or the cloud. In the following text, the recognition module and the detection module are embedded in the wearable device as an example, so as to make full use of the popularity and portability of the wearable device and further expand the application of the wearable device.

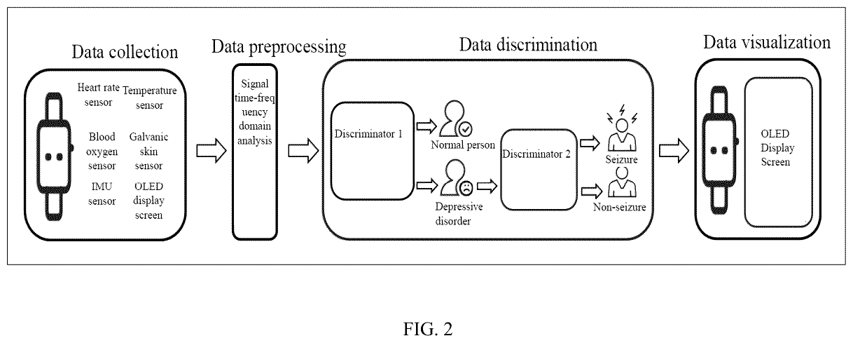

As shown in FIG. 2, the solution of the present application generally includes two parts: the wearable device and the model algorithm. Researchers can train the depressive disorder recognition model based on unsupervised transfer learning (or labeled as discriminator 1) and the depressive disorder seizure detection model based on few-shot learning (or labeled as discriminator 2) through their own data sets, and import these models into wearable devices. Then, the wearable device collects the user's heart rate, blood oxygen (two channel data can be obtained), galvanic skin, skin temperature signal and inertial measurement unit (IMU) signal data and preprocesses the data, and then carries out depressive disorder recognition and seizure detection analysis. Finally, the recognition and seizure detection results are displayed on the display screen of the wearable device, and meanwhile, the physiological data of the user can be calculated inside the wearable device, and the heart rate value, the blood oxygen value, the galvanic skin value, the epidermis temperature value and the like can be displayed.

### Embodiment of a Wearable Device

The wearable device provided by the application can be realized in various forms. FIG. 3 illustrates a schematic diagram of a wristband wearable device. The device includes an STM32 main control board, multiple sensors, and an organic light-emitting diode (OLED) display screen. The sensor type may be set as required, for example, a galvanic skin sensor, a body temperature sensor, a blood oxygen sensor, a pulse sensor, an inertial measurement unit (IMU) sensor, and the like.

In one embodiment, the STM32 main control board is configured to connect and communicate with multiple sensors as follows:
STM32 main control board and the galvanic skin sensor: read the value of the galvanic skin signal through an analog-to-digital converter (ADC) on the STM32 using an analog input pin;
STM32 main control board and the body temperature sensor: perform bidirectional communication with the body temperature sensor by using an I2C bus through SCL and SDA pins to read temperature data;
STM32 main control board and the blood oxygen sensor: perform asynchronous communication with the blood oxygen sensor by using a universal asynchronous receiver-transmitter (UART) serial port through TX and RX pins to read blood oxygen data;
STM32 main control board and the pulse sensor: perform full-duplex communication with the pulse sensor through the SCK, MISO, MOSI and CS pins by using the SPI bus to read the pulse data;
STM32 main control board and the myoelectric sensor: read the voltage value of the myoelectric signal through the ADC using the analog input pin;
STM32 main control board and the IMU sensor: perform bidirectional communication with the IMU sensor by using an I2C bus through the SCL and SDA pins to read acceleration, gyroscope and magnetometer data;
STM32 main control board and the OLED display screen: perform full-duplex communication with the OLED display screen by using the SPI bus through the SCK, MISO, MOSI and CS pins to send display commands and data; and
STM32 main control board and the Bluetooth module: asynchronously communicate with the Bluetooth module by using a UART serial port through TX and RX pins to transmit and receive wireless data.

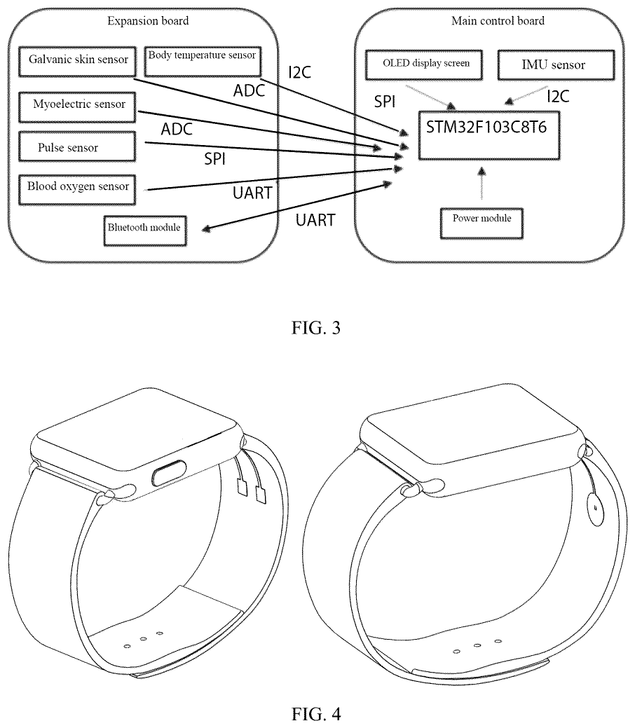

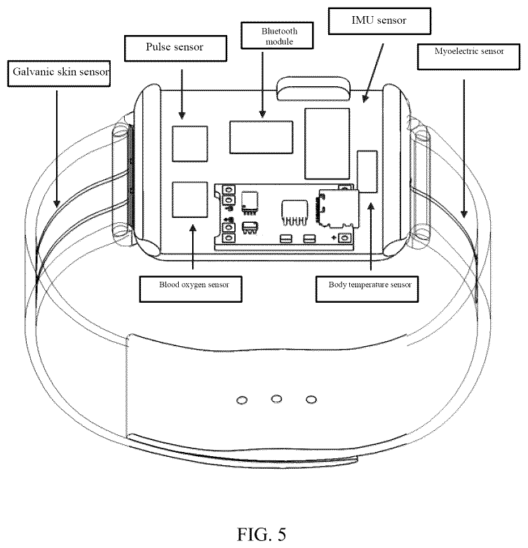

FIG. 4 illustrates an overall appearance view of a wristband wearable device. FIG. 5 illustrates an internal structure diagram of a wristband wearable device. The top of the device is equipped with an OLED display screen, which is used to display the heart rate value, blood oxygen value, galvanic skin value, skin temperature value calculated by the STM32 main control board and the results obtained by the depressive disorder recognition and seizure detection algorithm model in the wearable device. The galvanic skin sensor and myoelectric sensor can be embedded in the strap, connected to the internal main control board through the data line, and the body temperature sensor, blood oxygen sensor and pulse sensor are directly embedded under the device. In the process of detection, these sensors need to be close to the skin, and the display screen on the hardware device is used to display the physiological indicators and the results of depressive disorder recognition and seizure state detection in real time.

### Embodiment of Training a Depressive Disorder Recognition Model

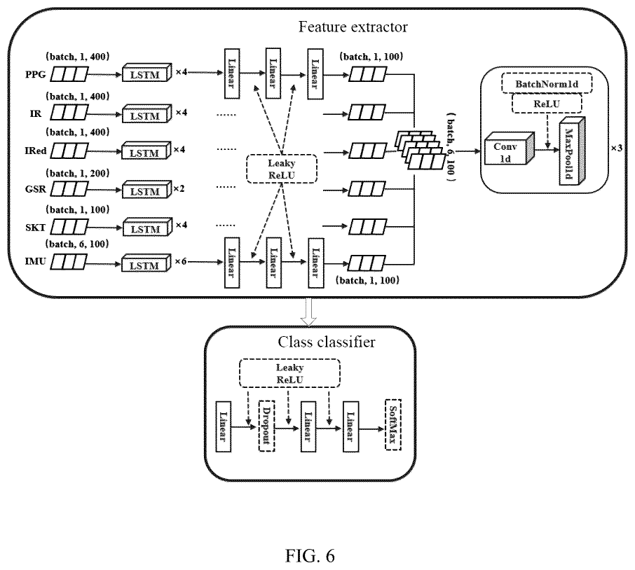

FIG. 6 illustrates a schematic diagram of the backbone network model structure of the depressive disorder recognition task, which generally includes a feature extractor and a classifier. The input multi-modal data includes: PPG is the pulse signal data, IR is the infrared light data in the blood oxygen signal data, IRED is the red light data in the blood oxygen signal data, GSR is the galvanic skin signal data, SKT is the skin temperature signal data, and IMU is the inertial measurement unit signal data. Because the input is the channel data with six time sequence characteristics and different physical meanings, the feature extraction is carried out on each channel data separately at the beginning of the model. In order to deal with the temporal information better, the LSTM layer (long short-term memory network) is introduced, and the linear layer is used to map the temporal features output by the LSTM layer to the target space. Then, six one-dimensional vectors of the same dimension are obtained and spliced in the first dimension. Then, the multi-modal data mining is carried out on the concatenated data by convolution, pooling and other operations, so as to further mine and express the complex correlation of different channel data. Finally, the features extracted by the feature extractor are transmitted to a class classifier consisting of a Linear layer, a Dropout layer and a Softmax output layer to obtain the probability of each class.

LSTM is used to extract the features of each channel data, which can learn and remember the long-term dependence relationship, and solve the problem that ordinary recurrent neural network (RNN) is difficult to capture long sequences. Moreover, the design structure of LSTM helps to maintain the gradient flow and to effectively solve the problem of gradient disappearance in the training process of traditional RNN. It should be noted that LSTM can also be replaced with gated recurrent unit (GRU).

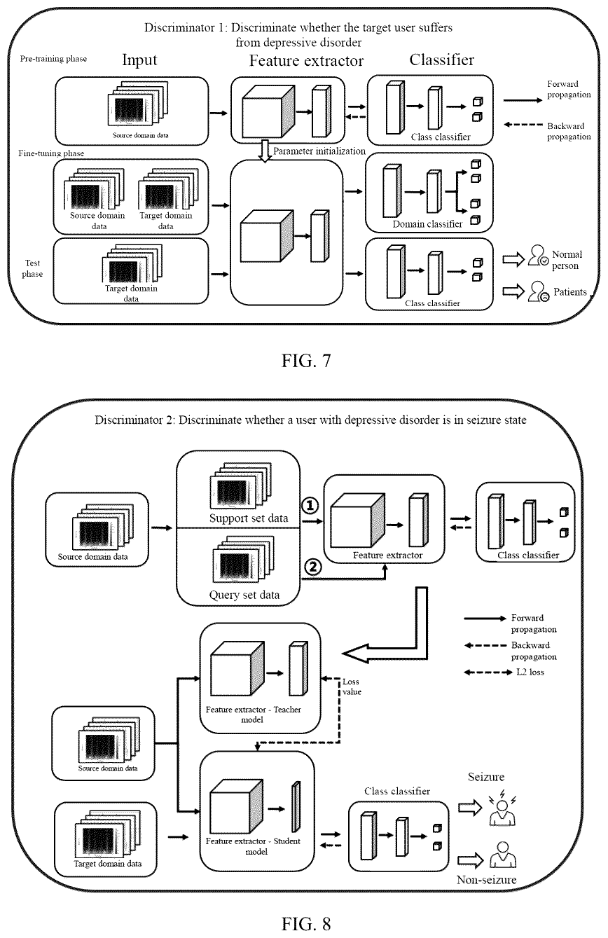

FIG. 7 illustrates a schematic diagram of a training process for a depressive disorder recognition model. For the depressive disorder recognition task, in one embodiment, unsupervised transfer learning is used for solving. A two-phase training process of pre-training and fine-tuning is adopted as a whole, and then the fine-tuned customized model of the target user can be used for testing or practical application.

Specifically, in the pre-training phase, the feature extractor and the class classifier are trained using the user data in the source domain (the source domain refers to the data exposed to the model in the training phase), and the cross-entropy loss function is used to assess the accuracy of the classification. The pre-trained feature extractor has learned a rich feature representation of the source domain, and the class classifier has a good classification ability for the task of depressive disorder recognition. However, for the user data in the target domain (the target domain refers to the data that has not been used or seen in the pre-training phase), the feature extractor after pre-training does not learn the data feature representation of the user in the target domain. Due to the characteristics of the depressive disorder recognition task, the labeled data in the target domain cannot be provided, so the feature extractor is further fine-tuned and the unsupervised transfer learning method is adopted. In the fine-tuning phase, the source domain data in the training phase (including the data of normal people and patients with depressive disorders) is used as one class of labels, and the target domain data is used as another class of labels to fine-tune the feature extractor and the domain classifier in the pre-training phase. The cross-entropy loss function can also be used in the fine-tuning process, but when the gradient is updated, the application performs gradient inversion between the feature extractor and the domain classifier, so that the domain classifier cannot distinguish whether the input data comes from the source domain or the target domain, thereby enabling the feature extractor to learn the feature representation of the target domain data. Finally, the feature extractor in the fine-tuning phase and the class classifier in the pre-training phase can be used to form a target domain user customized model to recognize the depressive disorder of the target domain user.

### Embodiment of Training a Depressive Disorder Seizure Detection Model

The depressive disorder seizure detection model and the depressive disorder recognition model may employ the same or different model structures, and the present application is not limited thereto.

FIG. 8 illustrates a schematic diagram of a training process for a depressive disorder seizure detection model. Aiming at the task of depressive disorder seizure detection, the structure of depressive disorder recognition model is adopted for the backbone network model structure, but the model parameters are not the same; meanwhile, few-shot learning and knowledge distillation technology are used, and the three-phase training process of pre-training, knowledge distillation and fine-tuning is adopted as a whole.

Specifically, first, in the pre-training phase, the user data in each source domain is divided into multiple tasks, the data in each task is only from the user data in one source domain, and the data in one task is divided into support set data and query set data. A model parameter updating process is as follows: Firstly, n tasks are randomly selected from a task data set, and the support set data in the tasks are respectively used to perform gradient updating on the model initialization parameters for m times; secondly, the query set data in the tasks are respectively used for calculating the loss of the model after the m times of gradient updating of the corresponding tasks, so as to obtain n task losses; and finally, the n task losses are added together to perform a gradient update on the initialization parameters of the model, so that a model parameter update is completed, and the pre-training can be completed by performing training for multiple times in this way.

In the knowledge distillation phase, only the feature extractor obtained in the pre-training phase is considered to be distilled, and only the source domain data is used. In one embodiment, the feature extractor in the pre-training phase is used as a teacher model, and one-dimensional vectors with the same dimensionality can be respectively obtained from the input data through the teacher model and the student model, and meanwhile, an L2 loss function is used to perform mean square difference on the two one-dimensional vectors with the same dimensionality to obtain a loss value, and then the gradient update is performed on the student model through the loss value. By using knowledge distillation technology, a student model with fewer model parameters and good performance can be obtained, thus improving the detection efficiency and ensuring the real-time requirements of the depressive disorder seizure detection task.

Next, considering that the model does not learn the data feature representation of the seizure and non-seizure states of users in the target domain, a small amount of labeled data in the target domain will be provided to carry out fine-tuning on the student model and the class classifier in the pre-training phase. In that fine-tuning phase, only a small amount of labeled data in the target domain is used, and a cross-entropy loss function is used for gradient updating. Finally, the fine-tuned student model and the class classifier are combined to form a user customized model in the target domain, and the user data in the target domain can be used for seizure detection of depressive disorder.

### Embodiment of Depressive Disorder Recognition and Seizure Detection Using Wearable Devices

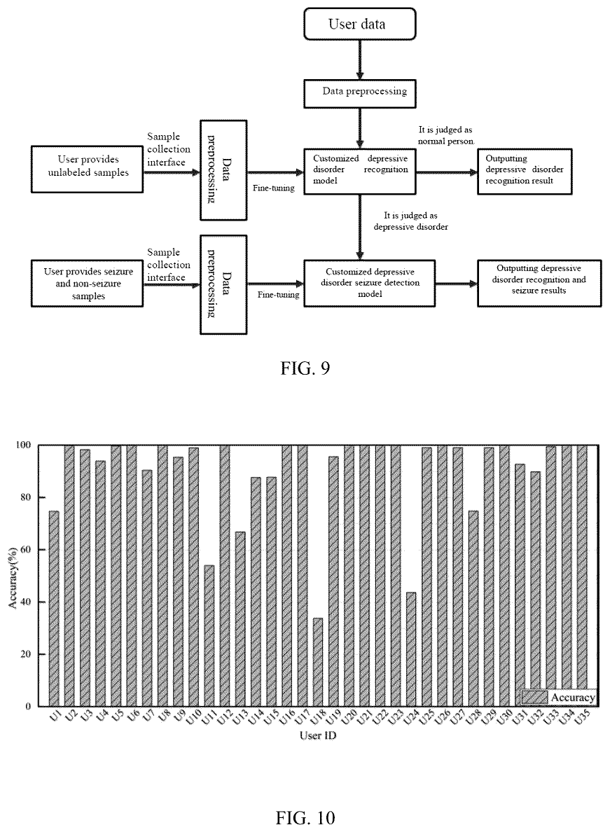

FIG. 9 illustrates a schematic diagram of a process of depressive disorder recognition and seizure detection using a wearable device. Firstly, the model of the preprocessing phase in the depressive disorder recognition task and the model of the knowledge distillation phase in the depressive disorder seizure detection task are embedded in the wearable device (or system), and the wristband wearable device can normally display the heart rate value, blood oxygen value, galvanic skin value, skin temperature value and the like of the data. When the user uses the device, the device is adjusted to the sample collection interface, and some unlabeled sample data is collected, and then a customized depressive disorder recognition model is obtained by fine-tuning. Then, the user data is continuously collected in a period of time, the data is sent to a customized depressive disorder recognition model after data preprocessing, if the proportion of depressive disorders determined by the system in the period of time is higher than a set threshold, the user is determined to be a depressive disorder patient, otherwise, the user is determined to be a normal person. Users who are determined to be depressive disorder patients are then asked to provide seizure and non-seizure sample data for fine-tuning to obtain a customized depressive disorder seizure detection model, so that the patient's status can be understood in real time. Finally, patients diagnosed with depressive disorders continuously collect data through wristband wearable devices, and send the data to the customized depressive disorder recognition model and the customized depressive disorder seizure detection model through data preprocessing. If one sample is determined to be a normal person, only the recognition of the depressive disorder is carried out, and the recognition result is output; and if one sample is determined to be a depressive disorder person, the seizure detection is carried out, and the recognition and the seizure result are output.

It should be noted that those skilled in the art can make appropriate changes or modifications to the above embodiments without departing from the spirit and scope of the present application. For example, for the preprocessing of the phase data set collected by the wearable device, various statistical features of the four physiological signals and the IMU signal, such as mean, variance, standard deviation, median, mode, maximum, and minimum, may also be used, and then these statistical features are used as substitutes for depressive disorder recognition and seizure detection. Meanwhile, these physiological signals can also be converted into time-frequency maps for processing, and the processed features can be used for depressive disorder recognition and seizure detection. For another example, the loss function used in the model training phase may also be of other types, such as the mean square error loss. In addition, the application is not limited to the recognition and seizure detection of depressive disorders, but is also applicable to different single psychological diseases such as anxiety disorders, mood disorders and the like, and can also be used for the recognition and seizure detection of the single psychological diseases by using related characteristics.

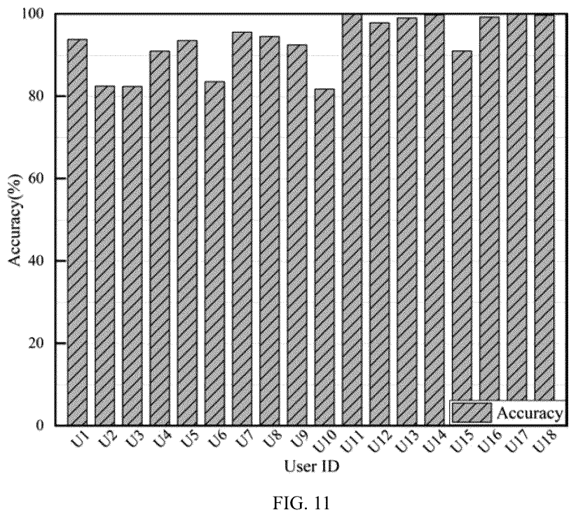

In order to further verify the effect of the application, an experiment is carried out. For the depressive disorder recognition task, the number of depressive disorder patients was 18 (corresponding to user No. U1-U18) and the number of normal persons was 17 (corresponding to user No. U19-U35), and the test accuracy of each person was obtained by the user leave-one-out method. The experimental results show that when the target users provide 5, 10, 20, 32 and 64 unlabeled samples, the average accuracy of depressive disorder recognition is 80.89%, 83.69%, 84.45%, 90.75% and 90.76%, respectively. For the task of depressive disorder seizure detection, the number of depressive disorder patients was 18 (corresponding to user No. U1-U18), and the test accuracy of each person was also obtained by the user leave-one-out method. The experimental results show that the average accuracy of depressive disorder detection was 93.15% when the target user provided 16 samples of non-seizure state and 16 samples of seizure state. FIG. 10 illustrates a schematic diagram of the accuracy rate of the depressive disorder recognition test for different users with 32 unlabeled samples. FIG. 11 illustrates a schematic diagram of the accuracy rate of the depressive disorder seizure detection test for different users with 16 non-seizure state samples and 16 seizure state samples.

In summary, the present application has the following advantages over the prior technology.

Firstly, the wearable device is combined with the model algorithm, the heart rate signal, the blood oxygen signal, the galvanic skin signal, the epidermis temperature signal and the IMU signal data of the user are acquired by the wearable device, the data are preprocessed and then transmitted to the algorithm model for depressive disorder recognition and seizure detection, the recognition and seizure results are obtained and displayed on the wearable device display screen. Meanwhile, the heart rate, blood oxygen, skin temperature and galvanic skin value can be calculated from the collected physiological data of the user and displayed on the display screen of the wearable device.

Secondly, the present application proposes a two-phase framework for the study of depressive disorders. In the first phase, the diagnosis of depressive disorder is carried out through the characteristics of multiple data fusion. In the second phase, when the user is diagnosed with depressive disorder, the seizure state is detected. This two-phase framework not only uses multi-mode physiological signals to recognize depressive disorders, but also detects the seizure of depressive disorders on this basis. By combining the dual functions of diagnosis and monitoring, users can understand their physical conditions in more detail.

Thirdly, in the task of diagnosing the depressive disorder, the recognition performance is improved in a mode based on unsupervised transfer learning, and the function of user personalization is realized; and when a new user uses the wearable device, the wearable device can be well adapted to the new user, so that the new user can accurately know the condition of the new user. Compared with the existing depressive disorder recognition scheme, the application realizes the personalized recognition function of the user, realizes cross-user by using a mode based on unsupervised transfer learning, and obviously improves the performance of the system.

Lastly, in the task of detecting the seizure of the depressive disorder, the detection performance is improved by means of the methods of small sample learning and knowledge distillation. A learning method base on a small sample enables that model to accurately recognize the seizure state of the depressive disorder; and based on the knowledge distillation technology, the model can be lightweight, so that the seizure state can be recognized more quickly, and the extreme harmful behavior of patients with depressive disorder can be effectively avoided. According to the application, the task of detecting the seizure state of the depressive disorder is realized for the first time, and the seizure state is quickly and accurately recognized by means of small sample learning and knowledge distillation, so that extreme harmful behaviors of a depressive disorder patient are effectively avoided.

The present application may be a system, method, and/or computer program product. The computer program product may include a computer readable storage medium that is uploaded with computer readable program instructions for causing a processor to implement various aspects of the present application.

A computer readable storage medium may be a tangible device that may hold and store instructions for use by an instruction execution device. A computer readable storage medium may be, for example but not limited to, an electronic storage device, a magnetic storage device, an optical storage device, an electromagnetic storage device, a semiconductor storage device, or any suitable combination of the foregoing. More specific examples (a non-exhaustive list) of the computer readable storage medium would include a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as a punch card or a raised structure in a groove on which instructions are stored, and any suitable combination of the foregoing. The computer readable storage medium used herein is not interpreted as an instantaneous signal itself, such as radio waves or other freely propagating electromagnetic waves, electromagnetic waves propagated through waveguides or other transmission media (e.g. optical pulses through fiber optic cables), or electrical signals transmitted through wires.

The computer readable program instructions described herein can be downloaded to various computing/processing devices from a computer readable storage medium, or to an external computer or external storage device over a network, such as the Internet, a local area network, a wide area network, and/or a wireless network. The network may include copper transmission cables, fiber optic transmission, wireless transmission, routers, firewalls, switches, gateway computers, and/or edge servers. A network adapter card or network interface in each computing/processing device receives computer readable program instructions from the network and forwards the computer-readable program instructions for storage in a computer readable storage medium in the respective computing/processing device.

The computer program instructions used to perform the operations of the present application may be assembly instructions, instruction set architecture (ISA) instructions, machine instructions, machine related instructions, microcode, firmware instructions, state setting data, or source or object code written in any combination of one or more programming languages, including object-oriented programming languages such as Smalltalk, C++, Python, and conventional procedural programming languages such as “C” language or similar programming languages. Computer readable program instructions can be executed entirely on the user's computer, partially on the user's computer, as a standalone software package, partially on the user's computer, partially on a remote computer, or entirely on a remote computer or server. In cases involving remote computers, the remote computer can be connected to the user's computer through any type of network, including a local area network (LAN) or a wide area network (WAN), or can be connected to an external computer (such as using an internet service provider to connect through the internet). In some embodiments, various aspects of the application are implemented by personalizing an electronic circuit, such as a programmable logic circuit, a field programmable gate array (FPGA), or a programmable logic array (PLA), with state information of computer readable program instructions that the electronic circuit may execute.

Various aspects of the present application are described herein with reference to flowchart illustrations and/or block diagrams of methods, apparatus (systems) and computer program products according to embodiments of the application. It should be understood that each box in the flowchart and/or block diagram, as well as combinations of boxes in the flowchart and/or block diagram, can be implemented by computer readable program instructions.

The computer readable program instructions may be provided to a processor of a general purpose computer, special purpose computer, or other programmable data processing apparatus to produce a machine, such that the instructions, when executed by the processor of the computer or other programmable data processing apparatus, resulting in means for implementing the functions/acts specified in the flowchart and/or block diagram block or blocks. The computer readable program instructions may also be stored in a computer readable storage medium that cause a computer, programmable data processing apparatus, and/or other devices to function in a particular manner, such that the computer readable medium storing the instructions comprises an article of manufacture, Which include instructions for implementing various aspects of the functions/acts specified in the flowchart and/or block diagram block or blocks.

The computer readable program instructions may also be loaded onto a computer, other programmable data processing apparatus, or other devices to cause a series of operational steps to be performed on the computer, other programmable data processing apparatus, or other devices, to produce a computer implemented process such that the instructions which execute on the computer, other programmable data processing apparatus, or other devices implement the function/act specified in the flowchart and/or block diagram block or blocks.

The flowchart and block diagrams in the figures illustrate the architecture, functionality, and operation of possible implementations of systems, methods and computer program products according to various embodiments of the present application. In this regard, each block in the flowchart or block diagrams may represent a module, segment, or portion of instructions, which comprise one or more executable instructions for implementing the specified logical function(s). In some alternative implementations, the functions noted in the blocks may occur out of the order noted in the figures. For example, two consecutive blocks may, in fact, be executed substantially concurrently, or they may sometimes be executed in the reverse order, depending upon the functionality involved. It also should be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions. It is well known to those skilled in the art that implementation by means of hardware, implementation by means of software, and implementation by means of a combination of software and hardware are all equivalent.

Various embodiments of the present application have been described above, and the above description is exemplary, not exhaustive, and is not limited to the disclosed embodiments. Many modifications and variations will be apparent to those of ordinary skill in the art without departing from the scope and spirit of the described embodiments. The terminology used in the application was chosen to best explain the principles of the embodiments, the practical application, or technological improvements in the marketplace, or to enable others of ordinary skill in the art to understand the embodiments disclosed herein. The scope of the application is defined by the claims.

What is claimed is:

1. A wearable depressive disorder recognition and seizure detection method, comprising the following steps of:
acquiring multi-modal data of a target user by using a wearable device;
inputting the multi-modal data into a trained depressive disorder recognition model to determine whether the target user is a depression disorder person or a normal person; and
inputting the multi-modal data to a trained depressive disorder seizure detection model to determine whether the target user is in a seizure state in case that the target user is determined as the depressive disorder person.

2. The method according to claim 1, wherein the depressive disorder recognition model comprises a first feature extractor and a first class classifier, and is trained according to the following steps of:
pre-training the first feature extractor and the first class classifier by using source domain data based on a first loss function, to obtain a pre-trained first feature extractor and a pre-trained first class classifier;
carrying out fine tuning on the pre-trained first feature extractor and a domain classifier by taking the source domain data used in the pre-training process as one class of labels and taking the target domain data as another class of labels to obtain a fine-tuned first feature extractor, wherein in the fine-tuning process, the first loss function is adopted to evaluate the loss, and to perform gradient inversion between the pre-trained first feature extractor and the domain classifier when the gradient is updated; and
using the fine-tuned first feature extractor and the pre-trained first class classifier to form a target domain user customized model as the trained depressive disorder recognition model.

3. The method according to claim 2, wherein the depressive disorder seizure detection model comprises a second feature extractor and a second class classifier, and is trained according to the following steps of:
pre-training the second feature extractor and the second class classifier using source domain data to obtain a pre-trained second feature extractor and a pre-trained second class classifier;
taking the pre-trained second feature extractor as a teacher model, performing knowledge distillation on the teacher model and a corresponding student model by using source domain data, wherein during the knowledge distillation process, the source domain data is passed through the teacher model and the student model to obtain one-dimensional vectors of the same dimension, and an L2 loss function is used to calculate an average square difference of these two one-dimensional vectors of the same dimension to obtain the loss value, and then the student model is then gradient updated based on this loss value;
carrying out fine-tuning on the student model obtained by knowledge distillation and the pre-trained second class classifier by using labeled data in the target domain to obtain a fine-tuned student model and a fine-tuned second class classifier; and
forming the fine-tuned student model and the fine-tuned second class classifier into a target domain user customized model as the trained depressive disorder seizure detection model.

4. The method according to claim 3, wherein the step of pre-training the second feature extractor and the second class classifier by using the source domain data comprises:
dividing user data of each source domain into a plurality of tasks, wherein the data in each task is only from user data of one source domain, and the data in one task is divided into support set data and query set data; and the second feature extractor and the second class classifier are pre-trained by using the support set data and the query set data to obtain the pre-trained second feature extractor and the fine-tuned second class classifier.

5. The method according to claim 2, wherein the first feature extractor comprises a long-short-term memory network layer, a first linear layer, a convolution layer and a pooling layer, and the first class classifier comprises a second linear layer, a Dropout layer and a Softmax layer;
the long-short-term memory network layer performs feature extraction on the data of each mode respectively; and
the first linear layer is used for mapping the time sequence characteristics output by the long-short-term memory network layer to a target space to obtain a plurality of one-dimensional vectors with the same dimension, splicing the one-dimensional vectors in the first dimension, sequentially transmitting the spliced data to the convolution layer and the pooling layer to obtain the fusion features of the multi-modal data, and transmitting the fusion features to the first class classifier.

6. The method according to claim 4, wherein the first feature extractor and the second feature extractor have the same or different structures, and the first class classifier and the second class classifier have the same or different structures.

7. The method according to claim 1, wherein the multi-modal data comprises pulse signal data PPG, infrared light data IR in blood oxygen signal data, red light data IRED in blood oxygen signal data, galvanic skin signal data GSR, skin temperature signal data SKT, and inertial measurement unit signal (IMU) data IMU.

8. A wearable depressive disorder recognition and seizure detection device, comprising:
a wearable device, configured for acquiring multi-modal data of a target user;
a recognition module, configured for inputting the multi-modal data into a trained depressive disorder recognition model to determine whether the target user is a depression disorder person or a normal person; and
a detection module, configured for inputting the multi-modal data to a trained depressive disorder seizure detection model to determine whether the target user is in a seizure state in case that the target user is determined as the depressive disorder person.

9. The device according to claim 8, wherein the recognition module and the detection module are embedded in the wearable device, and the wearable device comprises an STM32 main control board, a galvanic skin sensor, a body temperature sensor, a blood oxygen sensor, a pulse sensor, a myoelectric sensor, an inertial measurement unit (IMU) sensor, a display screen and a wireless module;
the STM32 main control board and the galvanic skin sensor read the value of the galvanic skin signal through an analog-to-digital converter (ADC) on the STM32 using an analog input pin;
the STM32 main control board and the body temperature sensor perform bidirectional communication with the body temperature sensor by using an inter-integrated circuit (I2C) bus through SCL and SDA pins to read temperature data;
the STM32 main control board and the blood oxygen sensor perform asynchronous communication with the blood oxygen sensor by using a universal asynchronous receiver-transmitter (UART) serial port through TX and RX pins to read blood oxygen data;
the STM32 main control board and the pulse sensor perform full-duplex communication with the pulse sensor through the SCK, MISO, MOSI and CS pins by using the serial peripheral interface (SPI) bus to read the pulse data;
the STM32 main control board and the myoelectric sensor read the voltage value of the myoelectric signal through the ADC using the analog input pin;
the STM32 main control board and the IMU sensor perform bidirectional communication with the IMU sensor by using an I2C bus through the SCL and SDA pins to read acceleration, gyroscope and magnetometer data;
the STM32 main control board and the display screen perform full-duplex communication with the display screen by using the SPI bus through the SCK, MISO, MOSI and CS pins to send display commands and data; and
the STM32 main control board and the wireless module asynchronously communicate with the wireless module by using a UART serial port through TX and RX pins to transmit and receive wireless data.

10. A non-transitory computer readable storage medium, having a computer program stored thereon, wherein the computer program, when executed by a processor, implements the steps of the method according to claim 1.
